(19) United States
(12) Patent Application Publication
(10) Pub. No.: US 2012/0302456 A1
(43) Pub. Date: Nov. 29, 2012

(54) VERTICAL FLOW-THROUGH DEVICES FOR MULTIPLEXED ELISA DRIVEN BY WICKING

(75) Inventors: George M. Whitesides, Newton, MA (US); Max Narovlyansky, Cambridge, MA (US); Ozge A. Halatci, Allston, MA (US); Charles R. Mace, Aubum, NY (US)

(73) Assignee: President and Fellows of Harvard College, Cambridge, MA (US)

(21) Appl. No.: 13/303,797

(22) Filed: Nov. 23, 2011

Related U.S. Application Data
(60) Provisional application No. 61/416,453, filed on Nov. 23, 2010.

(51) Int. Cl. C12M 1/40; G01N 21/78; C40B 30/04; C40B 40/10; G01N 33/53

(52) U.S. Cl. 506/9; 435/287.2; 506/18; 435/7.92

(57) ABSTRACT

The invention provides kits, methods and devices for detection of analytes in a biological sample. Capillary action is employed to carry out single or multiplexed immunoassays in a vertical flow-through format.

### CROSS-REFERENCE TO RELATED APPLICATIONS

This application is claims the benefit of priority to U.S. Provisional Application No. 61/416,453, filed Nov. 23, 2010, the entire disclosure of which is incorporated by reference herein.

### BACKGROUND

Portable, cost-effective diagnostic devices provide tools for unmet needs in resource-poor settings, from inexpensive quality control of agricultural products to healthcare.

Point-of-care (POC) devices, for example, lateral flow-through (LFT) immunoassays [1], or related immunochromatographic assays [2-5] have proven to be useful medical tools in resource-limited settings because they do not require equipment or trained personnel; the results of a given assay are produced rapidly and can be binary (yes/no) in nature.

Lateral-flow assays are typically used to detect the presence of a single analyte based on a single cut-off concentration that is chosen for each application (i.e., the cut-off concentration of human chorionic gonadotropin is about 50 pg/mL in a human pregnancy test). The lateral-flow format is, however, not ideal for multiplexed immunoassays because the spatial constraints of the device (and the visual read-out reagents) limits the number of analytes to be tested. The majority of commercially available devices test for only a single analyte (e.g., LFT pregnancy tests).

Multiplexed detection can facilitate differential diagnosis of common symptoms, which typically results in improved outcomes for patients [6]—whether by offering differential diagnosis or detecting simultaneous infections. Simultaneous detection also reduces the number of visits for differential diagnosis, reducing the number of visits to a physician, and the cost per diagnosis [6].

### SUMMARY OF THE INVENTION

The invention is based, in part, on the discovery that a capillary-driven vertical flow-through device can provide effective detection of analytes in a biological sample without the need for manual addition of multiple reagents, making it suitable for field use.

It is understood that any of the embodiments described below can be combined in any desired way unless mutually exclusive and that any embodiment or combination of embodiments can be applied to each of the aspects described below.

In one aspect, the invention provides a kit comprising: (a) a first tube comprising a first matrix spanning the cross-section of the inner tube and adapted to receive a biological sample; (b) a reagent membrane adapted for attachment to the first tube, the reagent membrane comprising a reagent that specifically binds a diagnostic marker; (c) a second tube comprising a wicking layer, the second tube adapted for attachment to the reagent membrane such that the reagent membrane is between the first tube and the second tube, wherein upon assembly of the first tube, the reagent membrane and the second tube into a device, such that when the device is placed in a vertical orientation the first tube is above the reagent membrane, when the first matrix is contacted with a liquid, capillary action drives the liquid from the first matrix through the reagent membrane into the wicking layer of the second tube; and (d) instructions for use.

In some embodiments, the first tube further comprises a second matrix comprising an antibody-enzyme conjugate, wherein the diagnostic marker specifically binds to the reagent and the antibody-enzyme conjugate specifically binds to the diagnostic marker.

In some embodiments, the reagent is conjugated to the reagent membrane or printed on the reagent membrane.

In some embodiments, the wicking layer comprises cellulose or cotton.

In some embodiments, the first matrix comprises blotting paper.

In some embodiments, the reagent for the diagnostic marker is an antigen.

In some embodiments, the reagent is an antibody-enzyme conjugate, an antibody-particle conjugate or an antibody-dye conjugate.

In some embodiments, a plurality of different reagents are conjugated to or printed on the same membrane.

In some embodiments, the reagents are arranged on the membrane in an array.

In some embodiments, the antibody-enzyme conjugate is a secondary antibody conjugated to alkaline phosphatase or horseradish peroxidase.

In another aspect, the invention provides a method for detecting the concentration of a diagnostic marker in a sample, the method comprising: (a) contacting a first matrix with a biological sample; (b) placing the first matrix with the biological sample into a diagnostic device, such that when the diagnostic device is in a vertical orientation, the first matrix is positioned higher than a wicking layer, wherein a regent membrane comprising a reagent that specifically binds a diagnostic marker is located between the first matrix and the wicking layer; (c) contacting the first matrix with a first solution, wherein capillary action causes the first solution to flow through the first matrix and the reagent membrane into the layer of wicking material; and (d) visually inspecting the reagent membrane.

In some embodiments, the method further comprises contacting the reagent membrane with a second solution containing a colorimetric substrate to provide a visual read-out of the results.

In some embodiments, the method further comprises contacting the reagent membrane with an antibody-enzyme conjugate, wherein the diagnostic marker specifically binds to the reagent and the antibody-enzyme conjugate specifically binds to the diagnostic marker.

In some embodiments, the reagent is an antibody-enzyme conjugate.

In some embodiments, the reagent is an antibody-particle conjugate or an antibody-dye conjugate.

In some embodiments, the wicking layer comprises cellulose or cotton.

In some embodiments, the first matrix comprises blotting paper.

In some embodiments, the reagent is an antigen.

In some embodiments, a plurality of different reagents are conjugated to or printed on the same membrane.

In some embodiments, the reagents are arranged on the membrane in an array.

### BRIEF DESCRIPTION OF THE DRAWINGS

FIG. 1A illustrates the distribution of reagents in the apparatus before and after the assay is performed according to one embodiment of the invention;

FIG. 1B illustrates removing the thimble and dipping into solution containing colorimetric substrate according to one embodiment of the invention;

FIG. 1C is a photograph of the bottom of the thimble after colorimetric development next to the tube with wicking material according to one embodiment of the invention;

FIG. 2 illustrates some embodiments of the types of possible results of indirect ELISA for detection of antibodies against Hepatitis C virus core antigen (HCVcAg) and rabbit serum against p41; and

FIG. 3 illustrates an embodiment in which an array of four 2-fold dilutions of two antigens and controls is spotted on a membrane; this array was tested with dilutions of rabbit antiserum from 1:125 to 1:4000 to obtain a range of responses.

### DETAILED DESCRIPTION OF THE INVENTION

As used herein, unless specifically indicated otherwise, the word “or” is used in the inclusive sense of “and/or” and not the exclusive sense of “either/or.”

In one aspect invention provides an analytical device, which uses capillary action and a matrix to carry out multiplexed immunoassays in a vertical flow-through format. In some embodiment, the matrix is a paper matrix. In some embodiments, the device is paper-based. In some embodiment, the device is disposable. In another aspect, the invention provides methods for designing such device. In another aspect, the invention provides methods for manufacturing such device.

In a specific embodiment, the invention provides a paper-based, disposable analytical device, which uses capillary action and a paper matrix to carry out multiplexed immunoassays in a vertical flow-through format.

Vertical flow-through can expand the capability of POC devices, which operate solely by capillary action. This configuration allows a two-dimensional array of supported reagents to be probed simultaneously. This design places a membrane, which is patterned with an array of antigens in the middle of the path of flow of liquid; capillary action drives the liquid downward, vertically through stacked paper and membrane. Manufacture of these two-dimensional arrays for multiplexed assays does require specialized equipment, but the resulting membranes can be mass-produced and shipped easily since they are light and require no refrigeration for preservation of desiccated analytes.

The ability to multiplex immunoassays provides a distinct advantage for inexpensive POC devices such as the device described herein. The use of a stack of layers of paper allows dry storage of antibodies for detection, and power-free pumping of liquid, resulting in the integrated and facile delivery of sample and reagents to a membrane for detection. A distribution of sample and antibodies pass through the membrane with the mobile aqueous phase, and rapidly bind to their corresponding antigens during this transition, allowing this immunochromatographic assay to be completed within a few minutes under kinetic, rather than thermodynamic, control of binding.

In some embodiments of the device and methods of using the device, an array of dilutions of antigens and controls can be interpreted by eye to calculate a simple measure (A), which measures the strength of immunological response. Using eight spots of p41 antigen on the membrane, greater than 100-fold range for detection of antibodies was obtained, indicating that printing dilutions of analytes provides a convenient way to expand the range of quantification based on corresponding antibodies.

In some embodiments, visual interpretation of multiplexed results is adequate for quantitative detection, especially using internal standards; the task of interpretation could be shared with medical specialists at a distant location using cell-phone based telemedicine [7]. In some embodiments, an enzyme-linked reagent is used to provide amplified colorimetric read-out.

The multiplexing capability of the device is demonstrated by performing indirect ELISA to assay two primary antibodies simultaneously, one specific for Hepatitis C virus core antigen (HCVcAg) and the second specific for the human immunodeficiency virus-1 p41 protein (HIV-1 p41). Dilutions of rabbit antiserum were tested against HIV-1 p41 and goat antibodies against HCVcAg.

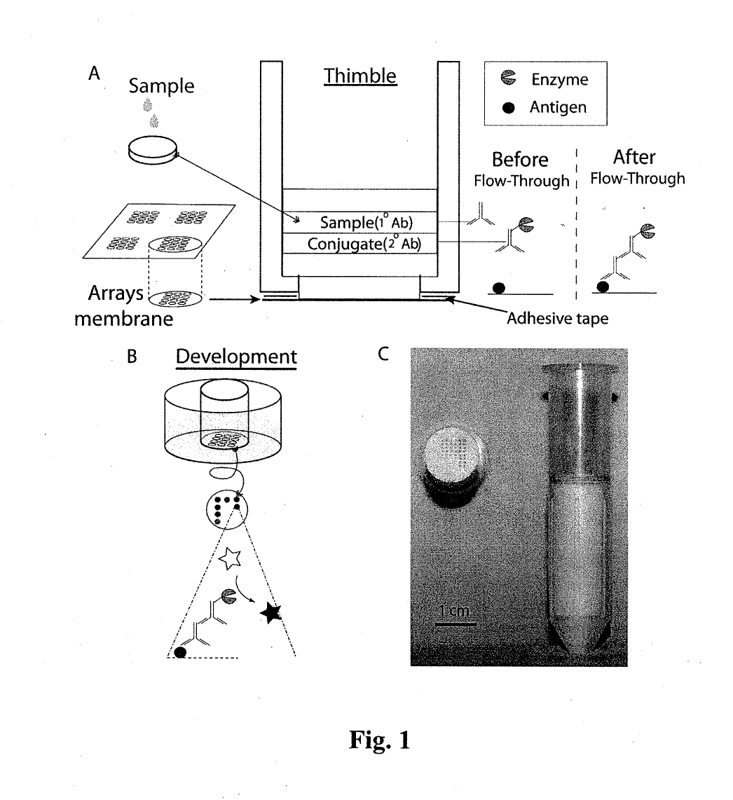

The schematic of FIG. 1A illustrates the distribution of reagents in the apparatus before and then after the assay is performed. A plastic cylinder (thimble) holds discs of stacked blotting paper, shown in cross-section as gray regions, above the membrane with immobilized antigens. These paper blotting discs are either empty (e.g., contain no reagents), or contain the antibody-enzyme conjugate (stored within the device), or contain the patient's sample, which can be added to the disc by the user and inserted into the thimble. The membrane supporting the array is attached to the bottom of the thimble by a non-migratory adhesive. This thimble fits within another plastic cylinder (wicking tube), that is partially filled with cellulose powder; the cellulose wicks the aqueous running buffer.

To the right of the thimble in FIG. 1A is a schematic illustration of the distribution of reagents before and after the flow-through is completed. Antigens (full circles) that are immobilized onto the membrane will bind to primary antibodies from the sample; these immune-complexes are then bound by a secondary antibody that is conjugated to an enzyme that catalyzes the formation of a chromophore (pac-man shape). After introduction of sample and running buffer by the user, the device carries out immunochromatography vertically through the membrane, drawn by the wick in the wicking tube.

To visualize the results, the thimble is removed and dipped into solution containing colorimetric substrate as illustrated in FIG. 1B. The image in FIG. 1C shows the bottom of the thimble after colorimetric development next to the tube with wicking material (cellulose powder held down by three discs of blotting paper). Secondary antibodies (against goat and rabbit immunoglobulins) conjugated with alkaline phosphatase (AP) to produce colorimetric signals for visual read-out.

In some embodiments, invention provides multiplexed antibody detection (i.e., simultaneous detection of antibodies against more than one antigen) in vertical flow-through format. In some embodiments, the device described herein is suitable for POC testing in the field that allows simultaneous probing of a high density of analytes on one membrane. Importantly, the device described herein eliminates the need for manual addition of multiple reagents. Moreover, the device described herein can be made suitable for field use.

In some embodiments, the device comprises high-density arrays that enable the use of internal standards for quantification.

The capabilities of vertical flow-through devices, demonstrated here, are able to function as point-of-care (POC) diagnostic systems for people that live in resource-limited settings or for clinical situations that require rapid decision-making (e.g. ambulatory and emergency-room care).

Applications for POC testing range from cardiovascular diseases [8] to infectious diseases, such as the pathogen responsible for human malaria [9]. Clearly, these portable diagnostic devices play an important and growing role in addressing healthcare needs in both developed and developing nations. POC tests also complement centralized medical facilities by alleviating some of the burden of testing dispersed populations. The availability of portable diagnostics allows the already-limited resources of centralized facilities to be allocated towards more sophisticated follow-up tests.

### 1. Operation of the Device

Two fitting plastic tubes that join to form a continuous path of flow that proceeds through the membrane were used. The upper half of the device (thimble) holds a membrane by a ring of adhesive (FIG. 1A). After the completion of the flow-through the user removes the thimble for colorimetric development and visual inspection of results.

Above the membrane, discs of blotting paper (pure cellulose) containing desiccated reagents (e.g., antibody-enzyme conjugates) were stacked. The bottom cylinder contains cellulose powder, which draws aqueous solutions vertically through the membrane by capillarity (illustrated in FIG. 1B). This device is designed to minimize the number of necessary manipulations: the user introduces a sample (i.e., whole blood or serum) and initiates the assay by adding buffer at the top of the device.

The reagent that is required for detecting an antigen or antibody (e.g., an enzymatic conjugate of secondary antibody) is adsorbed in one disc of blotting paper and stored in a dry state within the thimble. The user soaks a blank disc of blotting paper with a patient sample (e.g., 100 μl of biological fluid) and places the sample disc on top of a pre-assembled device. A running buffer is then added to the top of the device to initiate the immunoassay.

The material of the wick determines the rate of flow, according to Darcy flow. The material of the wick, with characteristic size and hydrophilicity [6] of pores that determine the meniscus of the fluid inside, defines the gradient of pressure due to the surface tension of water at the leading edge. The small pores in paper (stacked above cellulose powder) provide low permeability to pressure-driven flow. The flow in vertical flow-through device is independent of the volume or rate of addition of buffer ensuring consistent performance of the device for untrained users.

The running buffer elutes dried antibody conjugates from the discs of blotting paper. The liquid front also carries primary antibodies from the sample through an array of immobilized antigens for immunosorption. The pulse of eluted proteins passes through the membrane, resulting in simultaneous exposure of the entire membrane to mobile species.

Immunosorption is the binding of antibodies to their specific immobilized antigens and is the underlying principle of enzyme-linked immunosorbent assays (ELISAs). One clinical application of ELISAs is to detect a host immune response, i.e. the presence of primary antibodies developed within the host against antigens of a particular infectious agent. In indirect ELISA, enzyme-linked secondary antibodies bind to the primary antibodies recruited by the immobilized antigens; addition of enzyme substrate allows the generation of a signal for detection. In ELISAs, as the name implies, are enzyme-linked. In the scientific literature, however, this term is misused and applied to other methods of detection, such as fluorescence, electrochemical, etc.

An advantage of vertical flow-through described herein is that it results in kinetically controlled assays that give results much faster compared to assays in quiescent solutions, based on thermodynamic equilibrium. Conventional ELISAs typically suffer from limitation of diffusional mass-transport that is virtually absent in flow-through designs.

A volume of running buffer (approximately 4 mL) sufficient to carry the entire pulse of reagents through the membrane and to perform a subsequent wash is used. In some embodiments, this washing is used for achieving high sensitivity of detection, since it reduces non-specific binding of reagents, a feature which can be particularly important in flow-through assays which use relatively high concentrations of reagents relative to plate-based ELISA.

### 2. Selection of Visualization Reagent

Detection of fluorescence offers high sensitivity, but requires a power supply, and careful alignment of optics, which limit the portability of the device. For colorimetric detection, antibodies conjugated with gold colloids (or latex beads) provide good stability of assays, based on reagents stored dry on the device, but lack amplification, requiring a lot of these expensive reagents for detection.

Antibodies conjugated with enzymatic conjugates, such as horseradish peroxidase and alkaline phosphatase, e.g. ‘secondary’ antibodies for detection of human IgGs, are readily available and relatively inexpensive. In the final step of an immunoassay, these enzymes can be used to provide the colorimetric readout by catalyzing the conversion of a substrate from a colorless to a colored form. As described herein, the colorimetric readout is performed after the completion of immunochromatography, i.e. the running buffer has completely wicked through the thimble, by detaching the thimble and placing it into a solution containing substrate.

In some embodiments, a visual signal is produced by secondary antibodies conjugated with alkaline phosphatase (AP). AP is less susceptible to interference from the nitrocellulose matrix than similar conjugates, such as horseradish peroxidase [10]. A person of skill in the art will understand that other visualization reagents and visualization methods known in the art can be used instead.

### 3. Design of A Multiplexed Array

The ability to print multiple analytes on a membrane allows efficient detection of different markers of infections, while the use of dilution series yields quantitative information about the concentrations of these markers. The use of dilution series was previously demonstrated in lateral-flow format [11] using a series of parallel lines of diluted reagents, sprayed across a membrane at different distances downstream from the sample. Vertical flow-through places a membrane orthogonal to the direction of the flow of liquid, and, therefore, all the immobilized macromolecules are located approximately at the same distance downstream from the source of mobile species. This arrangement allows direct comparison of signals with internal standards from a single membrane.

Duplicate columns of each reagent (antigen or control) were printed using sequential two-fold dilutions for successive rows. For demonstration of detection of HCV and HIV-1, primary antibodies developed in goat and rabbit were used, respectively. Immunoglobulins (IgGs) developed in goat and rabbit were printed onto the membrane as controls. Secondary antibodies (against goat and rabbit IgG) bind to primary antibodies recruited by immobilized antigens, as well as control IgGs on the membrane. Traditional ELISA, performed in a plate-based format, involves dilution of the analyte (sometimes both analyte and antibodies for detection) to generate a response-curve. Printing a series of dilutions of antigens on the membrane can reduce the number of experiments, and helps to obtain as much information as possible using the minimal number of devices.

### 4. Choice of Materials

In some embodiments, materials for these diagnostic devices are chosen based on cost, weight and ease of disposal. For example, cellulose powder can be used as the wicking material because it is essentially fine sawdust, and can be produced inexpensively at local saw-mills.

Discs of bibulous paper were stacked within a plastic tube to sandwich a membrane supporting an array of antigens. Some of these discs above the membrane serve as a conduit for the running buffer; other discs contain antibodies for the immunoassay. Empty discs were utilized to pack the cellulose powder, thus ensuring good contact with the membrane for wicking

Stacking removes a major source of variability by using large areas of contact on both sides of the membrane. Simplification of the geometry of the flow-path increases the robustness of the assay, and obviates the need for adhesives that ensure contact between layers by attaching them to a plastic backing, such as in most commercial LFT assays.

The wetted discs of paper expand to fit even more tightly than before wetting within the plastic thimble, ensuring good contact between these softened layers within the stack.

Materials other than paper and cellulose can serve as wicking elements. Porosity and hydrophilicity are the primary properties that determine the rate of wicking The cellulose powder used provided a relatively slow rate of wicking (˜4 cm/10 min). Other types of materials, such as cotton, offer significantly slower rates of wicking than cellulose. The slower rates of wicking can increase sensitivity by allowing a longer time for antigen-antibody interaction [1]. The material downstream of the membrane determines the rate of wicking, thereby setting the balance between duration and sensitivity of the immunochromatographic assays.

Nitrocellulose (NC) was used as a membrane for immobilizing antigen arrays. NC is well-established for assays based on immunosorption, such as Western blotting, and is the typical chromatographic medium in lateral-flow assays. NC membranes allow for the facile immobilization of proteins due to non-covalent irreversible binding, which simplifies the production of microarrays. Other materials, such as poly(vinylidine difluoride) (PVDF) can also serve as the supporting matrix for arrays, but immobilization of proteins on PVDF membranes requires pre-wetting with methanol (REF). PVDF and nitrocellulose have low surface roughness compared with paper (pure cellulose), allowing printing of small spots and higher density arrays. Solid pins were used to transfer small volumes (100 nL) of solution to the membrane, resulting in spots with diameter of 0.5 mm, a size that can be readily resolved by eye. For spots of this size, our dime-size membrane can support an array of up to 400 spots, although a maximum of 100 spots were used in this example.

After use, the plastic pieces can be cleaned or recycled, while cellulose can be burned or used as compost, producing minimal waste.

### 5. Diagnostic Targets

Antibodies are valuable diagnostic markers of infection, especially for long-term, chronic infectious diseases. A pair of antibodies was detected for diagnosis of two chronic viral infections: HCV and HIV-1. Antibodies against p41 are highly diagnostic for HIV-1 [12] and HCVcAg has been developed as marker of hepatitis C infection. HCV is the most common type of Hepatitis infection, leading to chronic infections, liver cirrhosis and hepatocellular carcinoma. Chronic HCV represses the immune response of the patient by lowering T-cell count, and production of antibodies.

Other antibodies that are diagnostic markers of other infections can also be used to detect other infections such as pathogens responsible for human malaria.

Of course, the device and methods described herein can be adapted to detect other pathogens or disease markers using the knowledge and methods readily available to a person of ordinary skill in the art.

### 6. Multiplexed Indirect ELISA

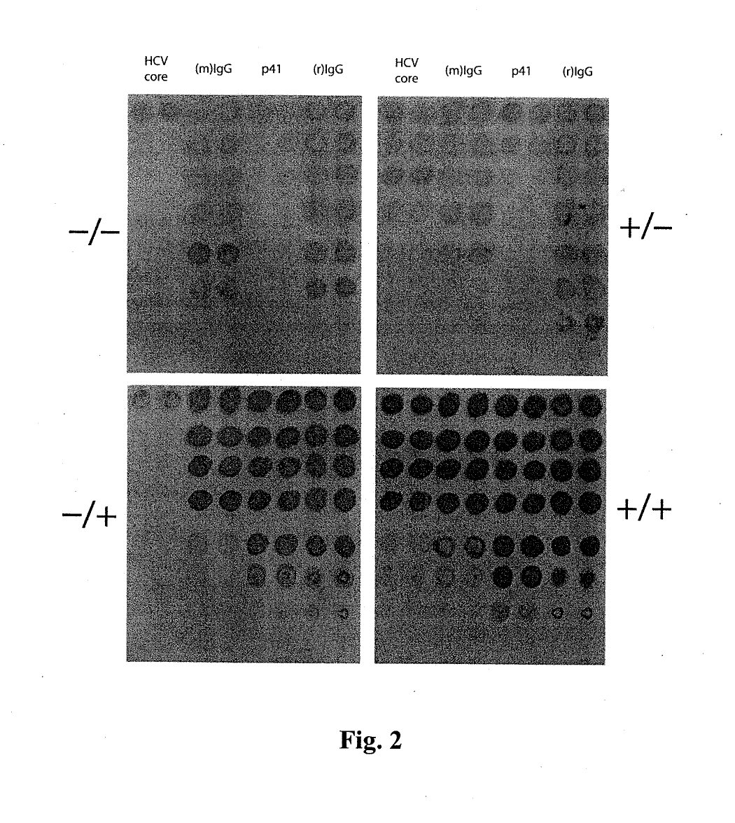

Some embodiments of the types of possible results of indirect ELISA for detection of antibodies against HCVcAg and rabbit serum against p41 are illustrated in FIG. 2.

FIG. 2 shows possible results of testing for HCV/HIV-1 infections with the methods of devices described herein. The membrane resulting from a sample lacking antibodies against HCVcAg or p41 illustrates no infections (−/−), where only the columns for positive controls of secondary antibodies produce signals. The (+/−) case illustrates the presence of HCV, but not HIV-1 antibodies. The (−/+) membrane would result from testing an individual with HIV-1 infection only, and the last membrane (+/+) illustrates the presence of HCV and HIV-1 co-infection. The membrane contained four pairs of duplicate columns of each reagent, and mouse and rabbit IgGs were used as controls. The maximum concentration of antigens in spotted solutions was 1 mg/mL (top row) with sequential two-fold dilutions for successive rows. The four panels in this figure are labeled depending on the presence or absence of primary antibodies against HCV/HIV-1 in the simulated sample.

The negative sample (−/−) showed strong signal from dilution series of controls. The (+/−) case shows detection of simulated “HCV-positive serum” (containing 500 ng/mL of mouse antibody against HCVcAg). The (−/+) case demonstrates detection of simulated “HIV-positive serum” (containing rabbit anti-p41 serum, approximately 200 ng/mL), and (+/+) indicates serum containing both antibodies, representing a co-infection with both viruses.

### 7. Interpretation of Colorimetric Results

Infection with either HCV or HIV-1 suppresses the immune system of the infected individual, resulting in variation in the strength of immune response, i.e., the concentration of specific antibodies in serum. This concentration varies with time since infection. Different concentrations of antiserum were used against p41 (serum of rabbit injected with p41 antigen) to simulate different concentration of primary antibody in serum.

Successive dilutions of rabbit antiserum were tested against p41 and detected the rabbit IgG using secondary goat anti-rabbit IgG conjugated with AP. As expected, successive dilutions of serum showed less intense signals from immobilized p41 antigens relative to rabbit IgG controls.

Dilution series of antigens were used to assess the status of immune response, i.e., the concentration of primary antibodies in the sample. An approximate concentration of primary antibodies was estimated based on the difference of intensities of antigens and controls.

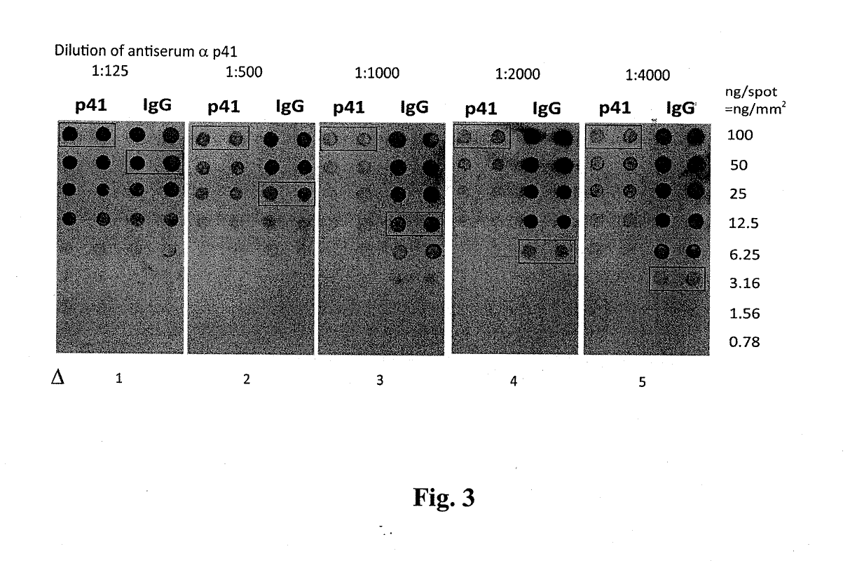

An array of four 2-fold dilutions of both antigens and controls was spotted on the membrane. This array was tested with dilutions of rabbit antiserum from 1:125 to 1:4000 to obtain a range of responses illustrated in FIG. 3. Higher concentrations of antibodies gave rise to visible signal from more rows of p41 antigens than of the control IgGs. Higher dilutions of serum model a weak immunological response against p41, such as can be the case for immunocompromised individuals, with advanced HIV infection, which is progressing into AIDS.

A rough gradation of the concentration of primary antibody was established by finding rows of comparable intensity at the top and bottom of the membrane, corresponding to antigens and controls. The number at bottom was subtracted from the number at the top. This difference was correlated (A between 1 and 5) with the 100-fold range of concentrations of primary antibodies (FIG. 3).

Determination of any concentration of p41 antigen and control IgG would require an accurate measurement of the intensities of color and a quantitative comparison. Using a dilution series of antigens and controls allows visual assessment by interpretation of intensity of color as either above or below an arbitrary cut-off. Vertical flow-through format used herein offers better internal standards that LFT format. The binary signals from individual spots at different dilution significantly expand the range of concentrations for detection of antibodies.

FIG. 3 shows detection of primary antibodies against p41 in diluted rabbit antiserum. The left half of each cropped sub-array contains dilutions of p41 antigen, while the right half contains rabbit IgG that serve as controls. Antigens and antibodies were spotted in duplicate columns with two-fold sequential dilutions of rows, starting from the top (the concentrations of both reagents on the membrane are indicated on the right). The maximum concentrations for both p41 and rabbit IgG on the membrane were 100 ng/spot (˜100 ng/mm²). Tests of dilutions of rabbit serum (dilution-factor is indicated above each array) resulted in different intensities from dilution-series of antigens (left) and controls (right) on each membrane.

Pairs of spots with similar intensities are outlined as rectangles in FIG. 3. To obtain a measure of concentration or titer of antibody in serum, the user subtracts the row numbers of antigens from the row number controls where spots of similar intensity occur to obtain a rough gradation of the strength of immune response, from strong (Δ=1) to weak (Δ=5).

Antibodies against a pair of antigens from HCV and HIV-1 antigens were detected using inexpensive devices based on vertical flow-through a membrane, demonstrating multiplexed detection, and quantification using internal standards. These results suggest that the vertical flow of a liquid through a vertically stacked set of materials is a useful platform for development of portable and cost-effective diagnostic immunoassays.

The patent and scientific literature referred to herein establishes knowledge that is available to those of skill in the art. The issued U.S. patents, allowed applications, published foreign applications, and references, including GenBank database sequences, that are cited herein are hereby incorporated by reference to the same extent as if each was specifically and individually indicated to be incorporated by reference.

This invention is further illustrated by the examples described herein, which should not be construed as limiting. Those skilled in the art will recognize, or be able to ascertain, using no more than routine experimentation, numerous equivalents to the specific substances and procedures described herein. Such equivalents are intended to be encompassed in the scope of the invention.

### REFERENCES

1. Wong, R. and H. Tse, Lateral Flow Immunoassay. 2008, New York City: Humana Press. 223.
2. Attrée, O., et al., Development and comparison of two immunoassay formats for rapid detection of botulinum neurotoxin type A. Journal of Immunological Methods, 2007. 325 p. 78-87.
3. Yuhi, T., et al., Resin-based micropipette tip for immunochromatographic assays in urine samples. Journal of Immunological Methods, 2006. 312 (1-2): p. 54-60.
4. Fung, K.-K., C.P.-Y. Chan, and R. Renneberg, Development of enzyme-based bar code-style lateral-flow assay for hydrogen peroxide determination. Analytica Chimica Acta, 2009. 634 (1): p. 89-95.
5. Lönnberg, M., M. Drevin, and J. Carlsson, Ultra-sensitive immunochromatographic assay for quantitative determination of erythropoietin. Journal of Immunological Methods, 2008. 339 (2): p. 236-244.
6. Price, C. P., Regular review: point of care testing. Brit. Med. J., 2001. 332: p. 1285-1288.
7. Martinez, A. W., et al., Simple Telemedicine for Developing Regions: Camera Phones and Paper-Based Microfluidic Devices for Real-Time, Off-Site Diagnosis. Anal. Chem., 2008. 80 (10): p. 3699-3707.
8. Dittmer, W. U. E., Toon H.; Hardeman, Willie M.; Huijnen, Willeke; Kamps, Rick; de Kievit, Peggy; Neijzen, Jaap H. M.; Nieuwenhuis, Jeroen H.; Sijbers, Mara J. J.; Dekkers, Dave W. C.; Hefti, Marco H.; Martens, Mike F. W. C., Rapid, high sensitivity, point-of-care test for cardiac troponin based on optomagnetic biosensor. Clinica Chimica Acta 2010. 411 (11-12): p. 868-873.
9. Stevens, D. Y., et al., Enabling a microfluidic immunoassay for the developing world by integration of on-card dry reagent storage. Lab on a Chip, 2008. 8 (12): p. 2038-2045.
10. Kolosova, A. Y., et al., Investigation of several parameters influencing signal generation in flow-through membrane-based enzyme immunoassay. Analytical and Bioanalytical Chemistry, 2007. 387 (3): p. 1095-1104.
11. Cho, J.-H. P., Se-Hwan, Semiquantitative, bar code version of immunochromatographic assay system for human serum albumin as model analyte. Biotechnology and Bioengineering, 2001. 75 (6): p. 725-732.
12. Hess, G., et al., Demonstration of antibodies to the surface (anti-p41) and core proteins (anti-p24) of the human immunodeficiency virus (HIV) in individuals positive for anti-HIV. Journal of Molecular Medicine, 1987 65 (13): p. 596-599.

What is claimed is:

1. A kit comprising:
(a) a first tube comprising a first matrix spanning the cross-section of the inner tube and adapted to receive a biological sample;
(b) a reagent membrane adapted for attachment to the first tube, the reagent membrane comprising a reagent that specifically binds a diagnostic marker;
(c) a second tube comprising a wicking layer, the second tube adapted for attachment to the reagent membrane such that the reagent membrane is between the first tube and the second tube,
wherein upon assembly of the first tube, the reagent membrane and the second tube into a device, such that when the device is placed in a vertical orientation the first tube is above the reagent membrane, when the first matrix is contacted with a liquid, capillary action drives the liquid from the first matrix through the reagent membrane into the wicking layer of the second tube; and
(d) instructions for use.

2. The kit of claim 1, wherein the first tube further comprises a second matrix comprising an antibody-enzyme conjugate, wherein the diagnostic marker specifically binds to the reagent and the antibody-enzyme conjugate specifically binds to the diagnostic marker.

3. The kit of claim 2, wherein the reagent is conjugated to the reagent membrane or printed on the reagent membrane.

4. The kit of claim 2, wherein the wicking layer comprises cellulose or cotton.

5. The kit of claim 2, wherein the first matrix comprises blotting paper.

6. The kit of claim 1, wherein the reagent for the diagnostic marker is an antigen.

7. The kit of claim 1, wherein the reagent is an antibody-enzyme conjugate, an antibody-particle conjugate or an antibody-dye conjugate.

8. The kit of claim 1, wherein a plurality of different reagents are conjugated to or printed on the same membrane.

9. The kit of claim 8, wherein the reagents are arranged on the membrane in an array.

10. The kit of claim 2, wherein the antibody-enzyme conjugate is a secondary antibody conjugated to alkaline phosphatase or horseradish peroxidase.

11. A method for detecting the concentration of a diagnostic marker in a sample, the method comprising:
(a) contacting a first matrix with a biological sample;
(b) placing the first matrix with the biological sample into a diagnostic device, such that when the diagnostic device is in a vertical orientation, the first matrix is positioned higher than a wicking layer, wherein a regent membrane comprising a reagent that specifically binds a diagnostic marker is located between the first matrix and the wicking layer;
(c) contacting the first matrix with a first solution, wherein capillary action causes the first solution to flow through the first matrix and the reagent membrane into the layer of wicking material; and
(d) visually inspecting the reagent membrane.

12. The method of claim 11, further comprising contacting the reagent membrane with a second solution containing a colorimetric substrate to provide a visual read-out of the results.

13. The method of claim 12, further comprising contacting the reagent membrane with an antibody-enzyme conjugate, wherein the diagnostic marker specifically binds to the reagent and the antibody-enzyme conjugate specifically binds to the diagnostic marker.

14. The method of claim 12, wherein the reagent is an antibody-enzyme conjugate.

15. The method of claim 11, wherein the reagent is an antibody-particle conjugate or an antibody-dye conjugate.

16. The method of claim 11, wherein the wicking layer comprises cellulose or cotton.

17. The method of claim 11, wherein the first matrix comprises blotting paper.

18. The method of claim 11, wherein the reagent is an antigen.

19. The method of claim 11, wherein a plurality of different reagents are conjugated to or printed on the same membrane.

20. The method of claim 19, wherein the reagents are arranged on the membrane in an array.
